(19) United States
(12) Patent Application Publication
(10) Pub. No.: US 2012/0045805 A1
(43) Pub. Date: Feb. 23, 2012

(54) SYSTEM AND METHOD FOR THE SYNTHESIS OF THERMOPLASTIC BIOPOLYMER

(76) Inventor: Mahdi Sadeghi Pour Marvi, Varamin (IR)

(21) Appl. No.: 12/860,932

(22) Filed: Aug. 22, 2010

(51) Int. Cl. C12P 7/42; C12M 1/00

(52) U.S. Cl. 435/146; 435/289.1

(57) ABSTRACT

The various embodiments herein provide a system and a method of synthesis of thermoplastic biopolymer such as poly-β-hydroxy butyric acid wherein the activated sludge and synthetic waste water are acclimatized in a sequential batch reactor. The diazotrophs are reacted with CO₂ in a secondary carbon source reactor. The acclimated activated sludge and the prepared diazotrophs are enriched for production of poly-β-hydroxy butyric acid in a polymer production reactor. The produced polymer is purified and collected. Herein, CO₂ is used as a secondary carbon source and is produced from a CO₂ generator. The diazotrophs used is azotobacter spp. Chorochome. The system comprises of a sequential batch reactor, a secondary carbon source reactor and a polymer production reactor.

### INTERNATIONAL FILING SPONSORSHIP STATEMENT

International filing for the present invention is sponsored by Iranian National Science Foundation.

### BACKGROUND

1. Technical field

The embodiments herein generally relate to the field of biopolymer production. The embodiments herein particularly relate to an incubator system which increases the production of a biopolymer. The embodiments herein more particularly relate to the synthesis of a biopolymer such as poly-β-hydroxy butyric acid based on a nitrogen fixation technology.

2. Description of the Related Art

Among the biodegradable polyesters, polyhydroxybutyrate (PHB) or poly-hydroxy butyric acid is the best-studied example. The biopolymer, poly-β-hydroxy butyric acid, belongs to the group of polyhydroxyalkanoates (PHAs). PHAs are synthesized by many bacteria and archaea as intracellular carbon and energy reserves. In the last decades, these biopolymers have received great attention due to their properties which resemble those of conventional petrochemical-based polymers. Their special physical traits like elasticity, high crystallization rate, biocompatibility, water resistance, oxygen impermeability and enantiomerically pure nature have broadened the scope of their utility in the field of industry and medicine. The production of PHAs from renewable resources like micro-organisms and their complete biodegradability give promising advantages from an environmental point of view.

Sequencing batch reactor (SBR) systems were used for the development of a system and operating procedures for the high production of polyhydroxyalkanoates (PHAs) by the wastewater treatment (activated sludge) bacterial cultures. It was found that unbalanced growth conditions stimulated massive PHA production in activated sludge biomass. Operating conditions had a significant effect on a PHA production and the composition of the accumulated copolymer. Fully aerobic (AE) conditions with nitrogen (N) and phosphorus (P) limitations were the optimum conditions for PHA production when the laboratory prepared mixtures of organics were used, while fully aerobic (AE) condition with the combinations of N, P, and potassium (K) limitations were better for PHA production using a high acetic acid industrial wastewater as the substrate. The use of activated sludge treating wastewater in which the sludge is acclimatized with municipal wastewater and acetate. PHA gets accumulated in the cell lines which are then recovered. Not only the wastewater, but the use of food waste has also been proposed for the production of PHA, in which the centrifuged food waste is put into Sequential Batch Reactor to produce PHA. But, the proposed methods are costly, time consuming and less efficient. So far, higher production costs of PHB have hindered the successful commercialization of PHB. Moreover, in all the proposed methods, only single carbon source is used.

Hence there is a need to provide an alternative to the conventional methods of producing petrochemical-based plastic materials which increases the production of PHA to achieve a commercial production of PHA using a nitrogen fixation technology.

The above mentioned shortcomings, disadvantages and problems are addressed herein and which will be understood by reading and studying the following specification.

### OBJECTIVES OF THE INVENTION

The primary object of the embodiments herein is to provide a method for the synthesis of poly-β-hydroxy butyric acid (PHB) using a nitrogen fixation technology.

Another object of the embodiments herein is to provide a process which utilizes a secondary carbon source apart from primary carbon source for enhanced production of PHB.

Yet another object of the embodiments herein is to provide a process of synthesizing PHB with increased efficiency and decreased cost of production.

Yet another object of the embodiments herein is to provide a process of synthesis of PHB with a minimal hazard to the environment.

Yet another object of the embodiments herein is to provide a process of synthesis of PHB which produces less green house gas than the polymer.

Yet another object of the embodiments herein is to provide a process of synthesis of PHB in which a carbon dioxide gas is used as a substitute for Nitrogen gas.

Yet another object of the embodiments herein is to provide a process wherein the hydraulic retention time is less.

Yet another object of the embodiments herein is to provide a process wherein the sludge retention time is high.

Yet another object of the embodiments herein is to provide an incubator system which uses Carbon dioxide gas for generating anoxia conditions.

Yet another object of the embodiments herein is to provide an incubator having a compact structure.

Yet another object of the embodiments herein is to provide an incubator having a high biomass concentration.

These and other objects and advantages of the embodiments herein will become readily apparent from the following detailed description taken in conjunction with the accompanying drawings.

### SUMMARY

The embodiments herein provide a process for the synthesis of poly-β-hydroxy-butyric acid based on nitrogen fixation technology. The reactor used for carrying out nitrogen fixation reactions is a secondary carbon source reactor or incubator. The use of carbon dioxide gas as a secondary source of carbon enhances the production of biopolymer in the micro-organisms. A suitable diazotroph is used for nitrogen fixation reactions. The medium used for culturing the microorganisms is nitrogen free medium with hydrophilic adjuvant and sucrose. The sucrose present in the medium is acting as a primary carbon source whereas the carbon dioxide gas in a relative concentration and at a special temperature conditions is acting as a secondary source of carbon to the micro-organisms. By using CO₂ as a secondary carbon source, the diazotrophs with low concentration of PHB become diazotroph with high concentration of PHB. The accumulated polymer is then recovered. The whole process takes around 14 days. The carbon dioxide relative concentration is 10% and the optimum temperature is around 24° C.

According to one embodiment herein, a method of synthesis of thermoplastic biopolymer comprises acquiring an activated sludge by treating a waste water and acclimatizing the activated sludge and a synthetic waste water in a sequential batch reactor for 60 days. A diazotrophs is pretreated with carbon dioxide in an incubator at a preset temperature for 30 days wherein the incubator is a secondary carbon source reactor and wherein the preset temperature is 24° C.;

The acclimated activated sludge taken from the sequential batch reactor is enriched with the pretreated diazotrophs taken from the incubator for 2 days in a polymer production reactor to produce a thermoplastic biopolymer wherein the thermoplastic biopolymer is poly-β-hydroxy butyric acid. The produced poly-β-hydroxy butyric acid purified and collected. The diazotroph used in secondary carbon source reactor is azotobacter spp. Chorochome. The carbon dioxide is a secondary source of carbon with a concentration of 10%.

According to one embodiment, a thermoplastic biopolymer production system comprises a sequential batch reactor, a secondary carbon source reactor and a polymer production reactor. The sequential batch reactor comprises housing with an inlet and outlet present on a top for adding an influent and withdrawing an effluent. A pump is connected to the inlet and the outlet for controlling a feed of the influent and a removal of the effluent. An air diffuser is positioned at a bottom for diffusing an air inside. An air pump is connected to the air diffuser for pumping the air to the air diffuser. A recirculation inlet and outlet are provided at a side wall surface. A recirculation pump is connected to the recirculation inlet and outlet for re-circulating the influents periodically. Two timers are provided for controlling a working of the recirculation pump and a working of the air pump respectively. A temperature controller is arranged for regulating a temperature within the sequential batch reactor. A sample outlet is provided for collecting a sample.

The secondary carbon source reactor comprises a metallic cubicle box arranged at a desired height level from a ground level. An inlet and an outlet are provided on a top of the metallic cubicle box for an addition and a removal of a material. A fine wood spontaneous media is arranged inside the metallic cubicle box for providing a medium for a reaction between the diazotrophs and CO₂ gas. A storage tank is positioned at the bottom of the metallic cubicle box for storing a solution containing diazotrophs. A recirculation pump is arranged for recirculating the solution containing diazotrophs from the storage tank to the fine wood spontaneous media via a plurality of tubes during the pre-treatment process of diazotrophs. A timer is provided for controlling a pumping time of the solution to the fine wood spontaneous media. A carbon dioxide generator is arranged for producing carbon dioxide gas during the pre-treatment process of diazotrophs. A carbon dioxide gas inlet is provided for an input of the produced carbon dioxide gas inside the secondary carbon source reactor. A water key is provided to manage an input of water into the storage tank and the secondary carbon source reactor for washing the secondary carbon source reactor after each use. A temperature controller is provided for controlling a temperature inside the secondary carbon source reactor. A sample outlet is arranged for collecting a sample.

The carbon dioxide generator comprises a firebox and a thermocouple. The solution containing the microorganism is an azotobacter medium with perlite used as a hydrophilic adjuvant.

The polymer production reactor comprises a chamber having an inlet on a top for adding materials. An air diffuser is positioned at a bottom for supplying air inside for aeration. An air pump is connected to the air diffuser for pumping an air to the air diffuser. A temperature controller is arranged for regulating a temperature inside the polymer production reactor. A pH controller is provided for adjusting the pH of the materials. An outlet is arranged for collecting a final product. The materials comprise of an acclimated activated sludge taken from the sequential batch reactor and pre treated diazotrophs collected from the secondary carbon source reactor.

According to one embodiment herein, a process for the production of poly-β-hydroxy-butyric acid involves three stages: firstly the preparation stage; secondly the reaction stage and thirdly the enrichment & purification stage. In the preparation stage, the materials are acclimated in a Sequential Batch Reactor (SBR) or Continuous stirred tank reactor (CSTR). In the reaction stage, the diazotrophs are reacted with CO₂ gas in an incubator or a secondary carbon source reactor (SCSR) for the production of PHB. In the enrichment & purification stage, the prepared micro-organisms are further cultivated for the production and accumulation of the biopolymer in a Polymer production reactor (PPR) or Plug flow reactor (PFR). Here, the cells become rich in biopolymer concentration. The biopolymer is then recovered and purified.

According to one embodiment herein, a process of synthesis of poly-β-hydroxy butyric acid wherein the activated sludge and synthetic waste water are acclimatized in a sequential batch reactor for around 60 days. The diazotrophs are reacted with carbon dioxide in a secondary carbon source reactor or incubator for around 30 days. Then the acclimated activated sludge taken from the sequential batch reactor and the prepared diazotrophs taken from the secondary carbon source reactor or incubator are enriched for production of poly-β-hydroxy butyric acid in a polymer production reactor. The produced polymer is then purified and collected. In the sequential batch reactor, the microorganisms present in the activated sludge are acting as diazotrophs. In secondary carbon source reactor, the azotobacter spp. Chorochome is acting as a diazotroph for carrying out the reactions. The carbon dioxide produced in the secondary carbon source incubator, is a secondary source of carbon used herein in a relative concentration of 10% and at a temperature of 24° C.

According to one embodiment herein, the secondary carbon source incubator system comprises of a sequential batch reactor, a secondary carbon source reactor or incubator and a polymer production reactor.

According to one embodiment herein, for the acclimation of the cells a sequential batch reactor is used. The medium is fed into the reactor. The medium comprises of activated sludge and synthetic wastewater. The reactor is operated in successive cycles of 4 hr each. Each cycle consists of different stages i.e. influent addition, aeration, anaeration, settlement and effluent withdrawal. The influent addition time is 5 min, aeration time is 120 min, anaeration time is 90 min, settlement time is 5 min and effluent withdrawal time is also 5 min. A timer controls the time period of each stage of the cycle. At the end of the aeration period and before settlement period, a defined volume of biomass is removed to keep the sludge retention time (SRT) of 10 days. Air is diffused inside by an air diffuser. The temperature of the reactor is kept 24° C. In order to maintain a homogenous distribution of substrate as well as uniform distribution of suspended biomass along the reactor depth, the liquid from the reactor is recirculated at the rate of 40 litres per day. The whole process of acclimation takes around 60 days.

According to one embodiment herein, for the reaction to occur, an incubator or a reactor called secondary carbon source reactor (SCSR) is used for incubation of bacteria. The SCSR is a metallic cubic box and is at a certain height from the ground. The micro-organisms or the diazotrophs are incubated for around 30 days. The medium/solution used for the cultivation of the diazotrophs is the azotobacter medium. The fresh medium/solution goes inside the incubator timely and replaces the expired medium. The medium/solution is stored in a source/container /storage. The temperature of the solution is maintained at 30° C. Pump solution system is provided which comprises an electrical pump and many tubs containing solution. The electrical pumps are provided to pump the solution from the source/container/storage to the incubator with the help of tubes. The bacteria are allowed to grow on the substrate made up of fine wood spontaneous media which is in the form of a plate. Timers are present for timely controlling the in and out passage of solution by turning on and off the electrical pump after every 15 min. The timers work as soon as the machine is turned on.

A Carbon dioxide generator is present for generation of Carbon dioxide gas. The generator comprises of a firebox, a thermocouple and controller for controlling the arrival of gas from the firebox to the incubator. The firebox produces a suitable amount of CO₂ which is relative to the concentration of biomass in the incubator. The CO₂ relative concentration herein is 10%. The temperature is maintained at 28-30° C. unless and otherwise noted and the initial pH is adjusted to 6.8 to 7. The whole process takes around 336 hrs or 14 days.

According to one embodiment herein, the prepared sludge from the sequential batch reactor and the prepared diazotrophs from the incubator are collected and fed into Polymer production Reactor for the production of biopolymer. Synthetic wastewater is added. Aeration is done for several additional hours till all the substrates are consumed by the bacteria and an endogenous phase of respiration is reached. Nutrients are added during aeration. Nutrients herein may comprise of carbon sources like glucose or sucrose. Acetate is also added as nutrient. Acetate is added in pulses separately from other nutrients. The acetate is added at least three times during each cycle. The first pulse of acetate is added after the first 15 min of aerobiosis to ensure a high Dissolved Oxygen (DO) concentration. The second and third pulses are added when the previous one is exhausted (i.e. after every interval of 1 hr). The temperature of the reactor is kept 24° C. Before 30 min of sampling the pH is adjusted to 6.5-7.0. Finally the solids are collected and centrifuged for 20 min.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a functional block diagram of the thermoplastic biopolymer production system according to one embodiment herein.

FIG. 2 shows a schematic functional diagram of Sequential Batch Reactor (SBR)/Continuous Stirred Tank Reactor (CSTR) in a thermoplastic biopolymer production system according to one embodiment herein.

FIG. 3 shows a schematic functional diagram of the Secondary Carbon Source Reactor (SCSR) or incubator in a thermoplastic biopolymer production system according to one embodiment herein.

FIG. 4 shows a schematic functional diagram of the polymer production reactor (PPR) or Plug flow reactor (PFR) in a thermoplastic biopolymer production system according to one embodiment herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide an incubator system comprising three reactors. For preparation and acclimation stage Sequential Batch reactor (SBR) is used. For reaction stage Secondary Carbon Source reactor (SCSR) or incubator is used. For enrichment and production stage Polymer production reactor (PPR) is used.

According to one embodiment herein, the sequential batch reactor comprises of housing with an inlet and outlet present on top for addition of influents and withdrawal of effluents. A pump is connected to the inlet and the outlet for controlling feed and removal of influents and effluents from the reactor. An air diffuser is positioned at the bottom of the reactor for diffusing air inside the reactor. An air pump is connected with the air diffuser for pumping the air to the diffuser. A recirculation inlet and outlet is connected to a recirculation pump for time to time re-circulation of the influents within the reactor. The timers are provided for timely controlling the working of the recirculation pump and the air pump within the reactor. A temperature controller for controlling temperature requirements in the reactor is provided. A sample outlet for timely collection of samples is present.

According to one embodiment herein, the secondary carbon source reactor or incubator comprises of a metallic cubic box with dimensions of 70×70×70 cm and is present at a height of 20 cm from ground level. An inlet and outlet is present on the top of the metallic cubic box for addition and removal of materials. A fine wood spontaneous media is present inside the cubic box for occurrence of reaction of microorganisms with CO₂ gas. Storage is positioned at the bottom of the cubic box for storing a solution for the growth of microorganisms. The solution comprises of an azotobacter medium with perlite used as a hydrophilic adjuvant. A recirculation pump is provided for replacement of exhausted solution with fresh solution containing microorganisms from the storage to the fine wood spontaneous media via tubes during the reaction. Timer is present for controlling the time of pumping of the solution to the fine wood spontaneous media. A carbon dioxide generator is present for generation of carbon dioxide gas during the reaction. The carbon dioxide generator comprises of a firebox and a thermocouple. The carbon dioxide gas generated is passed through an inlet inside the incubator. A water key is present to manage the entrance of water into the source and the incubator for washing the storage and incubator after each use. A temperature controller controls the temperature inside the incubator. Samples are taken out from sample outlet.

According to one embodiment herein, the polymer production reactor comprises of a chamber having an inlet on the top for addition of materials. The materials comprise of acclimated activated sludge taken from the sequential batch reactor and the diazotrophs reacted with carbon dioxide collected from the secondary carbon source reactor. An air diffuser is positioned at the bottom of the reactor for supply of air inside the reactor for aeration of substrates and microorganisms. An air pump for pumping the air to the air diffuser is present. A temperature controller controls the temperature inside the reactor. A pH controller for controlling the pH of the materials. Final product is collected from outlet.

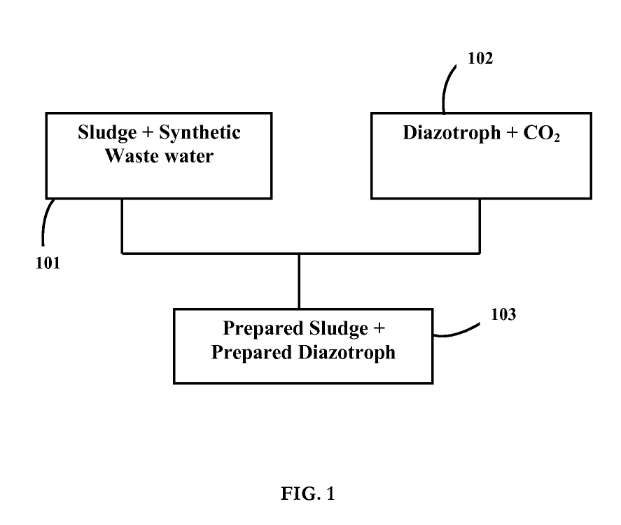

FIG. 1 shows the schematic functional diagram of the thermoplastic biopolymer production system according to one embodiment herein. With respect to FIG. 1, the complete incubator system comprises of three reactors: Sequential Batch Reactor 101, Secondary Carbon Source Reactor 102 and a polymer production reactor 103. According to FIG. 1, the activated sludge is acclimated with synthetic waste water in Sequential Batch Reactor 101 for around 60 days. The activated sludge is obtained from the purificator Co. of Tehran municipal Waste Water. This Co. produces activated sludge from the municipal waste water of Tehran city. The activated sludge is taken from the end of the aerobic step of the process of treatment of municipal wastewater. The composition of the synthetic wastewater used herein is described previously in Stockdale et al. 1968. The diazotrophs are reacted with CO₂ in Secondary Carbon Source Reactor 102 for around 30 days. The diazotrophs here is azotobacter spp. Chorochome. Then, the prepared sludge from the reactor 101 and the prepared diazotrophs from the reactor 102 are fed into the polymer production reactor 103 for enrichment and purification process. The enrichment and purification process takes around two days.

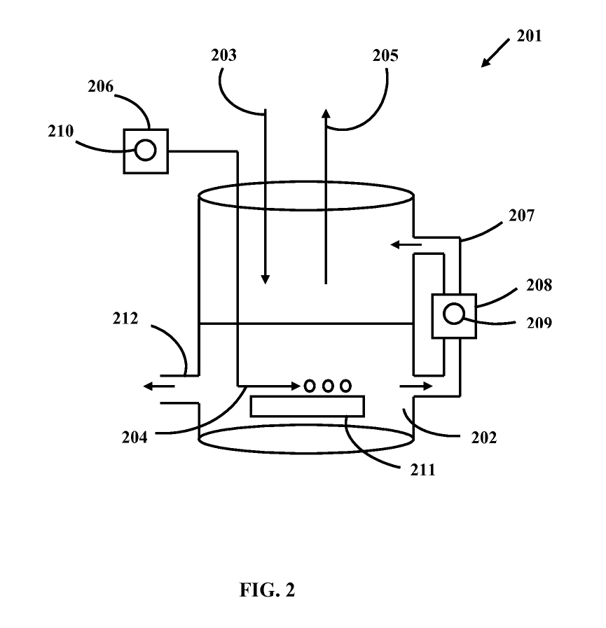

FIG. 2 shows the schematic functional diagram of Sequential Batch Reactor (SBR)/Continuous Stirred Tank Reactor (CSTR) in a thermoplastic biopolymer production system according to one embodiment herein. The Sequential batch reactors are a type of fill-and-draw reactors for activated sludge system waste water treatments. With respect to FIG. 2, the laboratory-scale sequencing batch reactor 201 is utilized to cultivate activated sludge. The reactor 201 is filled with the medium 202. The medium 202 comprises of Bark medium with synthetic wastewater and activated sludge. The reactor 201 is operated in successive cycles of 4 hr each. Each cycle consists of 5 min of influent addition from inlet 203, 120 min of aeration through air diffuser 204, 90 min of anaeration, 5 min of settling and 5 min of effluent withdrawal from the outlet 205. Air pump 206 pumps air into the reactor 201 through the air diffuser 204 present at the bottom of the reactor 201. A hydraulic retention time of 12 hr is maintained. The hydraulic retention time is a measure of the average length of the time that a soluble compound remains in a constructed reactor. Towards the completion of the aeration phase of 120 min and before the settling phase of 5 min, a defined volume of biomass is removed to keep a sludge retention time (SRT) of about 10 days. The sludge retention time is the average time till when the solids of activated sludge remain in the system. In a period of about 10 days, the whole medium gets replaced. The liquid is recirculated through recirculation pipe 207. Recirculation pump 208 circulates the liquid within the reactor 201. The liquid is recirculated to achieve a homogeneous distribution of substrate as well as uniform distribution of suspended biomass along the reactor depth. The recirculation also facilitates linear velocity, which restricts the existence of a concentration gradient during the whole process of the SBR operation. Timer 209 controls the recirculation pump. Timer 210 controls the air pump. Pumps are also present to control the medium feed and removal. The temperature controller 211 is present at the bottom of the reactor 201 to control the temperature. The temperature herein is kept at 24° C. Samples are timely taken out from the outlet 212.

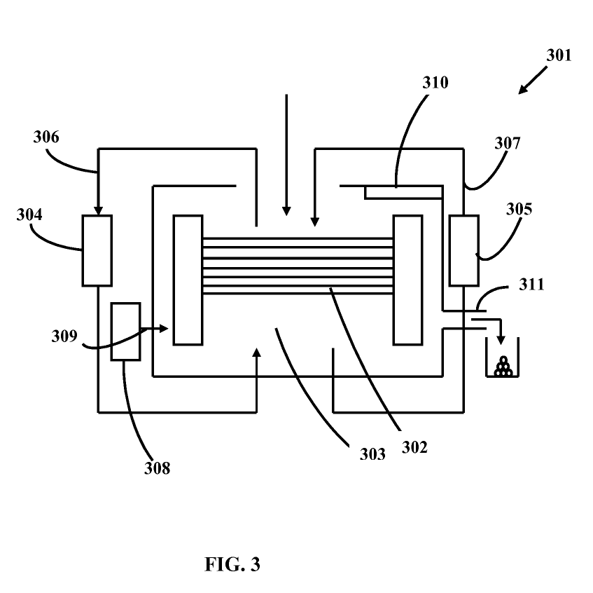

FIG. 3 shows the schematic functional diagram of the Secondary Carbon Source Reactor (SCSR) or incubator in a thermoplastic biopolymer production system according to one embodiment herein. With respect to FIG. 3, the SCSR or incubator 301 is a metallic cubic box with dimensions 70×70×70 cm present at a height of 20 cm from the ground. The incubator 301 is filled with medium 302 for cultivation of micro-organisms. The medium here is a fine wood spontaneous media in the form of a plate. The dimension of the plate is 50×50 cm. A storage/source/container 303 is present to store the solution. The solution is made up of azotobacter medium and water. The medium here is nitrogen free medium used to grow the diazotrophs. The solution is pumped through electric pumps 304 and 305 to the medium 302 for the cultivation of the diazotrophs. The temperature of the solution is maintained at 30° C. Table 1 shows the composition of the solution.

TABLE 1
SHOWING COMPOSITION OF THE SOLUTION

| S. No. | Ingredients | Quantity (g/l) |
|---|---|---|
| 1. | Carbon source (glucose) | 20 or 40 |
| 2. | MgSO₄•7H₂O | 0.4 |
| 3. | FeSO₄•7H₂O | 0.012 |
| 4. | Na₂MoO₄•12H₂O | 0.01 |
| 5. | K₂HPO₄ | 2 |
| 6. | NaCl | 0.4 |
| 7. | CaCl₂ | 0.11 |

Tubes 306 and 307 are present to circulate the liquid from the source 303 to the medium 302 for the reaction to occur. The tubes are around 10 mm in diameter. The fresh solution time to time replaces the exhausted solution. The glucose present in the solution act as a primary carbon source. A CO₂ generator 308 is present for generation of CO₂ in the incubator. The CO₂ herein is the secondary carbon source. The CO₂ generator consists of a firebox and a thermocouple. The firebox produces suitable CO₂ relative to the concentration of biomass in the incubator. The heat produced by the fire should not be more than 35° C. This may disturb the biochemical reactions taking place inside the incubator and affect the efficacy of incubator. The fire produced must be blue in color to indicate that the gas produced is CO₂. If the color of the fire is orange or yellow, then carbon monoxide is being produced and may hamper the biochemical reactions. If the temperature of the incubator increases cold water can be put on the CO₂ gas transfer tubes 309. The CO₂ relative concentration is 10%. The reaction takes place in the medium 302. Timers are present for controlling the time of the pumping the solution. The timer turns on and off the electrical pumps 304 and 305 after every 15 min as the machine is turned on. After every usage the source and the incubator should be washed properly for next usage. A water key is provided to manage the entrance of water to wash the source and the incubator after every usage. A temperature controller 310 is provided to control the temperature inside the incubator. A thermometer is used to measure the temperature inside the incubator medium. The temperature is maintained at 28-30° C. unless and otherwise noted and the initial pH is adjusted to 6.8 to 7. The total culture time is 336 hrs or 14 days. The final samples are taken out from the outlet 311.

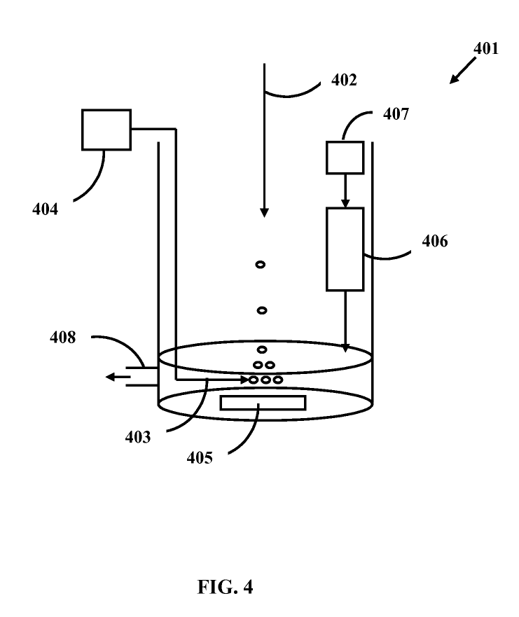

FIG. 4 shows the schematic functional diagram of the polymer production reactor (PPR) or Plug flow reactor (PFR) in a thermoplastic biopolymer production system according to one embodiment herein. The acclimated/prepared sludge with mixed liquor volatile suspended solids (MLVSS) of concentration of about 2000 mg/L is withdrawn from the SBR during the starvation period and fed in the Polymer production Reactor 401 from inlet 402. 0.5L of synthetic wastewater are added in the reactor 401 after the addition of prepared sludge and prepared diazotrophs. The reactor 401 is then aerated for several additional hours. The aeration is done through an air diffuser 403 present at the bottom of the reactor 401. The air is circulated to the air diffuser 403 from an air pump 404. The prepared diazotrophs are allowed to grow further for the enrichment process for getting enhanced production of PHB in the reactor 401. Various nutrients like glucose, sucrose and acetate are added during the aeration period. The sucrose is added in concentration of around 2500 ppm. The acetate is added in pulses separately from other nutrients and is added at least three times. Also, the glucose and acetate herein act as a substrate for the cultivation of microorganisms. For each supplement of carbon source, a volume of 10 ml of acetate solution (3,300 Cmmol/l) was added. The first pulse of acetate is added after the first 15 min of aerobiosis or aeration in order to ensure a high dissolved oxygen concentration. The second and third pulses are added when the previous one is exhausted (i.e. after every interval of 1 hr). The aeration is done till all the substrates and nutrients are consumed and an endogenous phase of respiration is reached. Then the aeration is stopped and 10 min after the beginning of the anaerobic phase the organic substrate is added through a capillary. As organic substrates acetate, acetate+glucose or only glucose is used. The temperature is kept at 24° C. with the help of temperature controller 405 present at the bottom of the reactor 401. Before 30 min of sampling the pH is adjusted to 6.5-7.0 with the help of pH controller 406 using H₂SO₄ 407. The final product is collected from the outlet 408 and washed for several hours with tap water.

The solids collected are centrifuged (5000×g) for 20 min and washed with distilled water. Cells lyses are done by methanol. The PHB is extracted with a solvent and separated from the remaining biomass. Samples are put in Oven (3.5 hr, 100° C.) until salvation is completed. Then the solvent is injected into water kept on shaker to get white precipitates of PHB. The precipitates are collected and dried. The powder obtained is transformed into pellets in an extruder.

### Experimental Data

### Stage 1—Acclimation and Preparation Stage

A laboratory-scale sequencing batch reactor (SBR) with a working volume of 8 litres was utilized to cultivate activated sludge. The experiments were performed with 4 litres Bark medium containing synthetic wastewater and 750 ml activated sludge from the end of the aerobic step of the wastewater. Activated Sludge achieved from purificator Co. of Tehran municipal Waste Water. This Co. produced Activated Sludge from municipal waste water of Tehran city. The reactor was operated in successive cycles of 4 hr each. One cycle consisted of 5 min of influent addition, 120 min of aeration, 90 min of anaeration, 5 min of settling and 5 min of effluent withdrawal. The hydraulic retention time was 12 hr. At the end of the aerobic period, before settling, a defined volume of biomass was removed to keep a sludge retention time (SRT) of 10 days. Air was introduced through an air diffuser for aeration from the bottom of the reactor. Timers controlled the recirculation, the air pump, and the pumps for medium feed and removal. The temperature was kept at 24° C. Recirculation at a rate of 40 litres/day was maintained throughout the investigation to achieve a homogeneous distribution of substrate as well as uniform distribution of suspended biomass along the reactor depth.

### Stage 2—Reaction Stage

### Microorganism and Growth Medium Preparation

The strain of Azotobacter chroococcum isolated from Tehran soils was used in this investigation. Burk nitrogen-free medium was prepared for cultivation of micro-organisms. The pH of the medium was adjusted to pH 7.0 with 1 M of HCl and 1 M of NaHCO₃. Table 2 shows the composition of the Burk nitrogen-free medium.

TABLE 2
SHOWING COMPOSITION OF THE MEDIUM

| S. No. | Ingredients | Quantity (g/l) |
|---|---|---|
| 1. | Carbon source (glucose) | 20 or 40 |
| 2. | MgSO₄•7H₂O | 0.4 |
| 3. | FeSO₄•7H₂O | 0.012 |
| 4. | Na₂MoO₄•12H₂O | 0.01 |
| 5. | K₂HPO₄ | 2 |
| 6. | NaCl | 0.4 |
| 7. | CaCl₂ | 0.11 |

### Solution Preparation

The inoculants of diazotrophs were prepared by addition of a nitrogen free media and a hydrophilic adjuvant in a fine wood spontaneous media. It is woody spontaneous materials. Perlite (mineral clay) was used herein as hydrophilic adjuvant and a carrier for the diazotroph. Vermiculite was also reported as suitable carrier for Azotobacter. pH of the inoculants was maintained at about 6.7 and contain CFU (Cell Forming Unite or number of cells/cm³ of inoculants) in the range of 10⁸. Table 3 shows the various characteristics of diazotroph inoculants used to conduct experiment.

SHOWING THE VARIOUS CHARACTERISTICS OF
DIAZOTROPH INOCULANTS

| Microorganism Species | Carrier | CFU | pH | Color |
|---|---|---|---|---|
| Azotobacter spp. | perlite | 10⁸ | 6.7 | yellow |
| Chorochome | | | | |

The medium (20 l per 1 m³ box) was inoculated with 4% (v/v) inoculums that had been pregrown for 24 hr in the medium and incubated at 30° C. for 60 days with vigorous aeration until solution were circulated during 15 min in intervals of 30 min in the box by motor pump. The incubator skeletal is a metal cubic box with dimensions 70×70×70 cm that it has high 20 cm from earth. Pump solution system consisted of an electrical pump and many tubs containing solution. The fine wood spontaneous media was used as a medium for cultivating the microorganism. The solution was contained in a container circulated in the incubator medium. In the end of every experiment, incubator was washed wholly for deleting prior process materials. The solution temperature was maintained at about 30 degree centigrade in this condition. CO₂ generator produced CO₂ for acting as a secondary carbon source. Temperature was maintained at 28-30° C. and the initial pH was adjusted to 6.8 -7. The bacteria were cultured for 336 hr (14 days). Glucose was the primary carbon source during 24 hr and CO₂ was secondary carbon source for rest of the 60 days of experiment.

### Stage 3—Enrichment and Purification Stage

The sludge with a mixed liquor volatile suspended solids (MLVSS) concentration of around 2000 mg/L was withdrawn from the SBR during the starvation period. After placing the sludge in the reactor, 0.5 l of the synthetic wastewater was added and aeration was supplied immediately. The sludge was aerated for several additional hours to reach the endogenous phase of respiration after consumption of all the substrates. Acetate was added in pulses separately from the other nutrients and this addition occurred three times during each cycle. The first pulse of acetate was added after the first 15 min of aerobiosis in order to ensure a high DO concentration. The second and third pulses were added when the previous one was exhausted (intervals of 1 h). For each supplement of carbon source, a volume of 10 ml of acetate solution (3,300 Cmmol/l) was added to a final concentration of 60 Cmmol/l. 10 min after the beginning of the anaerobic phase, the organic substrate was added through a capillary. As organic substrates acetate (107 mg C 1-1), acetate+glucose (each 53 mg C 1-1) or glucose (107 mg C 1-1) were used. In starting 1000 mg C/1 was used and 6 hr later 500 mg C/1 was used. The temperature was kept at 24° C. and the pH was adjusted 30 min before sampling to 6.5-7.0. Samples were withdrawn from the reactor, washed several times with tap water.

The solids collected were centrifuged for 20 min and washed with distillated water. Cells lyses were done by methanolyses process. PHB was extracted with chloroform as a solvent and separated from the remaining biomass. The samples were put in oven (3.5 h, 100° c) until salvation was completed. The solvent was injected into water and agitated on a shaker. The PHB precipitated as white powder of a purity of over 98%. The solvent was recycled in a closed system. The powder obtained in the extraction process was transformed into pellets in an extruder and analyzed for COD and stored biopolymer product.

### Experimental Result Analysis

A Rapid Gas Chromatographic method for the determination of poly-β-hydroxybutyric acid in microbial biomass was used.

### Chemicals

3-Hydroxybutyric Acid—The sodium salt of D and L-3-hydroxybutyric acid (Fluka, Buchs, Switzerland) was vacuum dried and kept over P₂O₅. Carrier gas (N₂), synthetic air and hydrogen for the GC flame ionization detector system (FID) were of 99.999% purity (Messer-Griessheim, FRG). All other chemicals used were of analytical grade quality (Merck, Darmstadt, FRG).

### Analytical Method

A computer-controlled gas chromatograph (Model HP 5840 A, Hewlett-Packard, USA) fitted as an all-glass system and equipped with a double FID was used for all analyses. In addition, an autosampler (Model HP 7671 A, Hewlett-Packard, USA) equipped with Hamilton syringes (Model 701 N, 10 gtl, Hamilton, USA) was employed to inject all samples.

### Column Materials

The internal diameter of all glass columns used was 0.1 in. An 8-ft column was filled with 2% Reoplex 400 (Merck, Darmstadt, FRG) on Chromosorb GAW 60 to 80 mesh whereas as a 6-ft column filled with Carbowax M 20 TPA on Chromosorb WAW DMCS 80 to 100 mesh was purchased directly from Hewlett-Packard, Vienna, Austria. All samples to be analyzed were prepared in screw-capped borosilicate glass test tubes with teflon seals (Coming Glassworks, New York, USA).

### Experimental Conditions

The 8-ft Reoplex column was used under the following conditions: initial temperature (T₁) of 90° C. and final temperature (T₂) of 150° C. The holding time for the initial temperature was 1 min and the holding time for the final temperature was 5 min. The rate of temperature increase was 8° C./min. The carrier gas flow rate was 30 ml N²/min. The 6-ft Carbowax column was employed at temperature 100° C. with a carrier gas flow rate of 20 ml N²/min. All other conditions corresponded to those used with the Reoptex-column.

### Preparation of Derivatives and Analysis of Pure Substances

All 3-hydroxybutyric acid samples were dissolved in 2 ml of acidified methanol containing 3% (v/v) H₂SO₄ and 1 ml of chloroform in a screw-capped test tube. The samples were then kept at 100° C. for 60 rain. After cooling to ambient temperature, 1 ml distilled water was added and the whole sample was then shaken for 10 rain. The two phases are permitted to separate. The organic phase is subsequently used for the GC analysis.

### Quantitative Determinations

Quantitative determinations were done by evaluating peak areas. PHB was quantitatively recovered to the extent of 100% in the form of the 3-hydroxybutyric acid methylester and 3-hydroxybutyric acid was used as a test substance. A linear relationship between peak areas was obtained. A concentration of up to 20/˜g of PHB in each sample were determined. It exhibited a correlation coefficient: r=0.999.

### Analysis of Cell Suspensions

The only pre-treatment of cell samples is simply centrifugation. The time for centrifugation can be considerably shortened by the addition of methanol to the suspension. This is done particularly when the cells contain large amounts of PHB. After decanting the liquid phase, the cells can be directly subjected to the acidic methanol treatment prior to GC analysis. Optimal conditions for this direct procedure were determined by parameter variation. Centrifuged cells are suspended in a mixture of 2 ml methanol (3% H₂SO₄, v/v) and 2 ml chloroform, and then heated at 100° C. for 3.5 hr. After cooling to room temperature, 1 ml H₂O was added and the sample was shaken vigorously for 10 min. The two phases are allowed to separate, during which cell detritus gathers at the interphase. The organic phase which is to be analyzed is placed in vials and can be stored unchanged at 4° C. for several weeks. The accuracy and reproducibility of this method can be increased as usual by employing an internal standard. Benzoic acid was found to meet all the requirements of an internal standard substance. The retention time of the benzoic acid methylester is approximately 2 rain longer than the retention time of the 3-hydroxybutyric acid methylester. For use as an internal standard benzoic acid is simply added to the acidified methanol reagent.

Within the linear range of the detector used on the GC apparatus, the results of this method for PHB determination depend neither on the volume of the cell suspension nor on the PHB content of the cells; the maximum standard deviation was ±0.5%. The reliability of the method is determined only by the accuracy of the measurement of the initial volume of the cell suspension. Shortening the time of treatment of the cell suspension at 100° C. reduces the sensitivity of the PHB determination; longer reaction times, however, lead to the formation of secondary products. As such they cause no difficulties, neither for the qualitative nor for the quantitative determination of the 3-hydroxybutyric acid methylester, but they do necessitate frequent heating of the columns. Table 4 shows the concentration of biopolymer obtained after experimentation.

SHOWING THE CONCENTRATION OF BIOPOLYMER
OBTAINED

| | | 3-hydroxy |
|---|---|---|
| | | butyric acid |
| S. No | Sample | (ppm) |
| 1. | Sludge 200 cc (Blank) | 0.0 |
| 2. | Sludge 200 cc + SCS 200 cc | 5.9 |
| 3. | Sludge 200 cc + SCS 200 cc + Waste Water 100 cc | 6.3 |
| 4. | Sludge 200 cc + Sodium Acetate 2500 ppm | 6.4 |
| 5. | Sludge 200 cc + Sodium Acetate 2500 ppm + SCS | 17.2 |
| | 200 cc | |
| 6. | Sludge 200 cc + Sucrose 2500 ppm | 7.9 |
| 7. | Sludge 200 cc + Sucrose 2500 ppm + SCS 200 cc | 326 |

*SCS—Secondary Carbon Source
cc—cubic cm
ppm—parts per million

From the table it can be concluded that the maximum concentration of 3-hydroxy butyric acid is obtained in presence of sucrose as primary carbon source and carbon dioxide as secondary carbon source (SCS). The obtained precipitates of the polymer are of 98% purity.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method of synthesis of thermoplastic biopolymer comprises:
acquiring an activated sludge by treating a waste water;
acclimatizing the activated sludge and a synthetic waste water in a sequential batch reactor for 60 days;
pretreating a diazotrophs with carbon dioxide in an incubator at a preset temperature for 30 days wherein the incubator is a secondary carbon source reactor and wherein the preset temperature is 24° C.;
enriching the acclimated activated sludge taken from the sequential batch reactor with the pretreated diazotrophs taken from the incubator for 2 days in a polymer production reactor to produce a thermoplastic biopolymer wherein the thermoplastic biopolymer is poly-β-hydroxy butyric acid;
purifying and collecting the produced poly-β-hydroxy butyric acid.

2. The method according to claim 1, wherein the diazotroph used in secondary carbon source reactor is azotobacter spp. Chorochome.

3. The method according to claim 1, wherein the carbon dioxide is a secondary source of carbon with a concentration of 10%.

4. A thermoplastic biopolymer production system consisting of:
a sequential batch reactor;
a secondary carbon source reactor; and
a polymer production reactor.

5. The system according to claim 4, wherein the sequential batch reactor comprises:
a housing with an inlet and outlet present on a top for adding an influent and withdrawing an effluent;
a pump connected to the inlet and the outlet for controlling a feed of the influent and a removal of the effluent;
an air diffuser positioned at a bottom for diffusing an air inside;
an air pump connected to the air diffuser for pumping the air to the air diffuser;
a recirculation inlet and outlet provided at a side wall surface;
a recirculation pump connected to the recirculation inlet and outlet for re-circulating the influents periodically;
two timers for controlling a working of the recirculation pump and a working of the air pump respectively;
a temperature controller for regulating a temperature within the sequential batch reactor; and
a sample outlet for collecting a sample.

6. The system according to claim 4, wherein the secondary carbon source reactor comprises:
a metallic cubicle box arranged at a desired height level from a ground level;
an inlet and an outlet present on a top of the metallic cubicle box for an addition and a removal of a material;
a fine wood spontaneous media arranged inside the metallic cubicle box for providing a medium for a reaction between the diazotrophs and CO₂ gas;
a storage tank positioned at the bottom of the metallic cubicle box for storing a solution containing diazotrophs;
a recirculation pump for recirculating the solution containing diazotrophs from the storage tank to the fine wood spontaneous media via a pluralities of tubes during the pre-treatment process of diazotrophs;
a timer for controlling a pumping time of the solution to the fine wood spontaneous media;
a carbon dioxide generator for producing carbon dioxide gas during the pre-treatment process of diazotrophs;
a carbon dioxide gas inlet for an input of the produced carbon dioxide gas inside the secondary carbon source reactor;
a water key to manage an input of water into the storage tank and the secondary carbon source reactor for washing the secondary carbon source reactor after each use;
a temperature controller for controlling a temperature inside the secondary carbon source reactor; and
a sample outlet for collecting a sample.

7. The system according to claim 6, wherein the carbon dioxide generator comprises a firebox and a thermocouple.

8. The system according to claim 6, wherein the solution containing the microorganism is an azotobacter medium with perlite used as a hydrophilic adjuvant.

9. The system according to claim 5, wherein the polymer production reactor comprises:
a chamber having an inlet on a top for adding materials;
an air diffuser positioned at a bottom for supplying air inside for aeration;
an air pump connected to the air diffuser for pumping an air to the air diffuser;
a temperature controller for regulating a temperature inside the polymer production reactor;
a pH controller for adjusting the pH of the materials;
an outlet for collecting a final product.

10. The system according to claim 9, wherein the materials comprises of an acclimated activated sludge taken from the sequential batch reactor and pre treated diazotrophs collected from the secondary carbon source reactor.
